(19) United States
(12) Patent Application Publication
(10) Pub. No.: US 2022/0257218 A1
(43) Pub. Date: Aug. 18, 2022

(54) ULTRASONIC DOPPLER BLOOD FLOW IMAGING METHOD AND SYSTEM

(71) Applicant: YUNNAN UNIVERSITY, Kunming (CN)

(72) Inventors: Yufeng ZHANG, Kunming (CN); Bingbing HE, Kunming (CN); Zhiyao LI, Kunming (CN); Hao LI, Kunming (CN); Xun LANG, Kunming (CN); Junhua ZHANG, Kunming (CN)

(21) Appl. No.: 17/618,014

(22) PCT Filed: Sep. 28, 2020
(86) PCT No.: PCT/CN2020/118271

§ 371 Date: Dec. 10, 2021

(30) Foreign Application Priority Data
Mar. 10, 2020 (CN) 202010162200.2

(51) Int. Cl. A61B 8/08; A61B 8/06

(52) U.S. Cl. CPC A61B 8/5276; A61B 8/5223; A61B 8/488; A61B 8/06

(57) ABSTRACT

An ultrasonic Doppler blood flow imaging method and system are provided. The method includes: acquiring a blood flow velocity measuring interest region and a number of transmitting sub-apertures; determining, according to the number of transmitting sub-apertures, inclination angle of a plane wave in each of the transmitting sub-aperture; determining, according to inclination angle, array element excitation delay time in each of the transmitting sub-aperture; controlling, according to array element excitation delay time, all of transmitting sub-apertures to synchronously transmit plane waves, and receiving echo signals with a full aperture; generating a radio frequency signal sequence according to echo signals; extracting blood flow Doppler signals of blood flow velocity measuring interest region according to radio frequency signal sequence; determining a blood flow velocity according to blood flow Doppler signals; and generating a Doppler blood flow image of blood flow velocity measuring interest region according to blood flow velocity.

### CROSS REFERENCE TO RELATED APPLICATION

The present application claims priority to the Chinese Patent Application No. 202010162200.2, filed with the China National Intellectual Property Administration (CNIPA) on Mar. 10, 2020, and entitled “ULTRASONIC DOPPLER BLOOD FLOW IMAGING METHOD AND SYSTEM”, which is incorporated herein by reference in its entirety.

### TECHNICAL FIELD

The present disclosure relates to the technical field of ultrasonic Doppler blood flow imaging, and in particular, to an ultrasonic Doppler imaging blood flow method and system.

### BACKGROUND ART

With the rapid development of modern society, smoking, dyslipidemia, obesity, unhealthy diet and lack of physical exercise and other common bad habits lead to frequent occurrence of atherosclerosis. Meanwhile, the incidence of atherosclerosis-induced vascular diseases is also increasing year after year. Cardio-cerebrovascular diseases, represented by myocardial infarction (MI) and cerebral ischemic stroke, can lead to high disability and mortality due to the short effective rescue time upon incidence. Hence, as a major public health problem that may affect the social development, the cardio-cerebrovascular diseases have become the biggest threat to our life and health. Clinically, monitoring the course of the atherosclerosis enables effective prediction for the occurrence of the cardio-cerebrovascular diseases for early intervening treatment. Till date, main methods such as the computed tomography angiography (CTA), magnetic resonance imaging (MRI), digital subtraction angiography (DSA) and ultrasound diagnosis have been available to clinically detect the atherosclerosis. The CTA, MM and DSA can develop the vascular lesion, accurately detect the vascular geometry, and determine a risk level of the atherosclerosis according to the vessel wall thickness and the vessel distortion. However, the above three detection methods cannot be used to monitor the atherosclerosis for a long time because of the prohibitive cost and the radiation to human bodies. The ultrasound diagnosis technologies can detect the progression of the atherosclerosis according to measured hemodynamic information to diagnose the cardio-cerebrovascular diseases as early as possible, and has the advantages of no radiation, low price, timeliness and so on.

Hemodynamics is based on the blood flow velocity distribution. According to the blood flow velocity distribution, hemodynamic parameters such as shear stress, velocity shear rate and wall shear rate can be calculated. Due to viscosity of the blood and friction between the blood flow and the vessel wall, the blood flow velocity distribution in healthy blood vessels is in a form of a parabola. The velocity at a center of the lumen in the radial direction is maximum and gradually decreases with further closer to the vessel wall. By contrast, in atherosclerotic diseased blood vessels, the blood flow velocity distribution changes due to the influence of plaques. It is no longer the parabola on which the velocity in the center is maximum and gradually decreases on two sides, and even shows a turbulence and eddy current. Therefore, accurate detection on the blood flow velocity distribution is of great significance to prevention and treatment of the cardio-cerebrovascular diseases. There are two main ultrasound technologies used to obtain the blood flow velocity distribution, that is, speckle tracking and Doppler ultrasound. The speckle tracking respectively tracks blood speckles at different radial positions in two consecutive frames of B-mode ultrasound images to acquire the whole blood flow velocity distribution from the upper vessel wall to the lower vessel wall. Since the treatment time of the speckle tracking technology lags the clinical signal acquisition, the blood flow velocity distribution cannot be displayed in real time, and the measurement results are susceptible to speckle noise, the speckle tracking hasn't been widely used in clinic. The Doppler ultrasound technology estimates the blood flow velocity based on a principle of measuring the Doppler frequency shift with the reflection method, and has the advantages of fast processing velocity and high measurement accuracy. However, as the maximum detectable velocity is restricted by a pulse repetition frequency, the velocity aliasing may occur.

Presently, the Doppler ultrasound technology widely used in clinic includes pulsed Doppler duplex scan imaging and color Doppler blood flow imaging. The pulsed Doppler duplex scan imaging combines B-mode ultrasound imaging and pulsed Doppler to display the surrounding tissue structure and the spectrum of the blood flow Doppler signal at the same time, and achieves the dual functions of range and velocity measurements. Because the pulse repetition frequency is partially sacrificed for B-mode ultrasound imaging, the maximum blood flow velocity that this technology can detect is small. The color Doppler blood flow imaging displays a two-dimensional (2D) color blood flow image on the B-mode ultrasound image synchronously while scanning the blood flow on multiple spatial positions. This technology uses the color brightness to indicate the velocity, and red and blue colors to indicate directions of the velocity. Compared with pulsed Doppler, the color Doppler blood flow imaging shows the blood flow velocity distribution more intuitively in space. However, in order to ensure a frame rate of color blood flow imaging, the number of pulses transmitted at each scanning position is limited, the time for observing the blood flow is short, and the signal-to-noise ratio (SNR) is low. Therefore, this technology is only used for qualitative analysis on the blood flow velocity. In summary, the two commonly used Doppler ultrasound technologies are restricted by the pulse repetition frequency.

In order to solve the above problem, ultrafast ultrasound has been proposed, targeting mainly plane-wave imaging. The full aperture is used to transmit the ultrasonic signal, and the echo signal of the entire imaging region can be obtained through a single transmission. The pulse repetition frequency is equal to the frame rate, which is as high as 20,000 frames per second. Due to the lack of a transmit focus, the SNR of the echo signal is low and the imaging quality of the single plane-wave (SPW) algorithm is poor. The coherent plane-wave compounding (CPWC) algorithm is proposed to improve the imaging quality of the SPW. The algorithm obtains multiple frames of plane-wave images successively from the same imaging region at multiple angles by changing the transmitting angle of an ultrasound transducer, obtains a time sequence for multiple frames of plane waves tilted at different angles, and coherently superimposes the multiple frames of images in the sequence to obtain a compounded image. Since the CPWC averages the multiple frames of images, it can effectively smooth the noise and improve the SNR. Using different transmitting angles, it can effectively solve the lack of an edge of the imaging object. However, as the CPWC averages the time sequence for the multiple frames of plane waves tilted at different angles, the loss of the pulse repetition frequency is doubled while the SNR is improved. In addition, in the time sequence for the multiple frames of plane waves tilted at the different angles, the blood speckles move constantly to result in motion artifacts in the compounded image, thereby hindering the measurement of the blood flow velocity.

To sum up, existing plane-wave algorithms applied to the ultrafast ultrasonic Doppler blood flow imaging mainly include: the SPW, the CPWC, and the multi-angle plane-wave repeated compounding algorithm based on the recursive technology.

As one frame of radio frequency signal can be generated whenever the plane wave is transmitted, the SPW is defective mainly in that the radio frequency signal is greatly interfered by the noise for no transmit focus and the low SNR.

In order to overcome the main defect of the SPW, the CPWC generates a synthetic focus by superimposing multi-angle plane waves to improve the imaging quality. As the coherently superimposed multi-angle plane waves are transmitted successively, the CPWC is defective mainly in that multiple frames of radio frequency signals are superimposed to generate one frame of compounded radio frequency signal to double the loss of the pulse repetition frequency.

The multi-angle plane-wave repeated compounding algorithm based on the recursive technology is proposed to overcome the main defect of the CPWC. For multiple frames of radio frequency signals compounded each time, other frames than the first frame are repeatedly used for the next compounding, so the algorithm improves the pulse repetition frequency of the CPWC to be the same as that of the SPW. However, the coherently superimposed multi-angle plane waves are still the multi-angle plane waves transmitted one by one, causing the low pulse repetition frequency. Therefore, the multi-angle plane-wave repeated compounding algorithm based on the recursive technology fails to address the motion artifacts in the compounded radio frequency signal, and has the low pulse repetition frequency.

### SUMMARY

An objective of the present disclosure is to provide an ultrasonic Doppler blood flow imaging method and system, to maximize the pulse repetition frequency, and suppress the motion artifacts in the compounded radio frequency signal during ultrafast ultrasonic Doppler blood flow imaging.

In order to achieve the above objective, the present disclosure employs the following technical solutions: an ultrasonic Doppler blood flow imaging method includes:

acquiring a blood flow velocity measuring interest region and a number of transmitting sub-apertures;

determining an inclination angle of a plane wave in each of the transmitting sub-apertures according to the number of the transmitting sub-apertures;

determining array element excitation delay time in each of the transmitting sub-apertures according to the inclination angle;

controlling, according to the array element excitation delay time, all of the transmitting sub-apertures to synchronously transmit the plane wave, and receiving an echo signal with a full aperture;

generating a radio frequency signal sequence according to the echo signal;

extracting a blood flow Doppler signal of the blood flow velocity measuring interest region according to the radio frequency signal sequence;

determining a blood flow velocity according to the blood flow Doppler signal; and

generating a Doppler blood flow image of the blood flow velocity measuring interest region according to the blood flow velocity, where the Doppler blood flow image is configured to display blood flow velocities at different spatial positions in the blood flow velocity measuring interest region.

In some embodiments, the determining an inclination angle of a plane wave in each of the transmitting sub-apertures according to the number of the transmitting sub-apertures may specifically include:

determining, when the number of the transmitting sub-apertures is an even number, the inclination angle of the plane wave in each of the transmitting sub-apertures according to a formula

$${β}_{n} = \{ \begin{matrix}- arctan ( \frac{y}{\frac{1}{2} ( N - 2 n + 1 ) \times {L}_{s u b} \times {W}_{e l e m e n t}} ) & 1 \leq n \leq \frac{N}{2} \\ arctan ( \frac{y}{\frac{1}{2} ( 2 n - N - 1 ) \times {L}_{s u b} \times {W}_{e l e m e n t}} ) & \frac{N}{2} < n \leq N\end{matrix} ,$$

where, βₙ is the inclination angle of the plane wave in each of the transmitting sub-apertures; y is a vertical coordinate of the blood flow velocity measuring interest region; N is the number of the transmitting sub-apertures; n is a serial number of the transmitting sub-aperture, 1≤n≤N; L_{sub} is the number of array elements in each of the transmitting sub-apertures; and Wₑₗₑₘₑₙₜ is a width of an array element; and

determining, when the number of the transmitting sub-apertures is an odd number, the inclination angle of the plane wave in each of the transmitting sub-apertures according to a formula

$${β}_{n} = \{ \begin{matrix}- arctan ( \frac{y}{\frac{1}{2} ( N - 2 n + 1 ) \times {L}_{s u b} \times {W}_{e l e m e n t}} ) & 1 \leq n < \frac{N + 1}{2} \\ 90 & n = \frac{N + 1}{2} \\ arctan ( \frac{y}{\frac{1}{2} ( 2 n - N - 1 ) \times {L}_{s u b} \times {W}_{e l e m e n t}} ) & \frac{N + 1}{2} < n \leq N\end{matrix} .$$

In some embodiments, the determining array element excitation delay time in each of the transmitting sub-apertures according to the inclination angle may specifically include:

determining the array element excitation delay time in each of the transmitting sub-apertures according to a formula

$$t ( {l}_{s u b} ) = \frac{( {l}_{s u b} - 1 ) \times {W}_{e lement} \times sin ( 90 ° - {β}_{n} )}{c} ,$$

where, t(l_{sub}) the array element excitation delay time; c is an ultrasound transmission velocity (UTV) in tissues; and l^{sub} is a serial number of an array element.

In some embodiments, the generating a radio frequency signal sequence

performing beamforming on the echo signal with an ultrasound delay-and-sum (DAS) method to generate a frame of compounded radio frequency signal for the blood flow velocity measuring interest region; and

generating the radio frequency signal sequence according to the compounded radio frequency signal.

In some embodiments, the determining a blood flow velocity according to the blood flow Doppler signal may specifically include:

determining the blood flow velocity according to a formula

$$v = \frac{c {f}_{p}}{4 π {f}_{0}} \times φ ,$$

where, v is the blood flow velocity; φ is a phase shift of the blood flow Doppler signal; fₚ is a pulse repetition frequency, which equals to a frame rate of the compounded radio frequency signal; and f₀ is a central frequency of an ultrasound transducer.

An ultrasonic Doppler blood flow imaging system includes:

an acquisition module for a blood flow velocity measuring interest region and the number of transmitting sub-apertures, configured to acquire the blood flow velocity measuring interest region and a number of transmitting sub-apertures;

an inclination angle determination module, configured to determine an inclination angle of a plane wave in each of the transmitting sub-apertures according to the number of the transmitting sub-apertures;

an array element excitation delay time determination module, configured to determine array element excitation delay time in each of the transmitting sub-apertures according to the inclination angle;

a synchronous transmission module, configured to control, according to the array element excitation delay time, all of the transmitting sub-apertures to synchronously transmit the plane wave, and receive an echo signal with a full aperture;

a radio frequency signal sequence generation module, configured to generate a radio frequency signal sequence according to the echo signal;

a blood flow Doppler signal extraction module, configured to extract a blood flow Doppler signal of the blood flow velocity measuring interest region according to the radio frequency signal sequence;

a blood flow velocity determination module, configured to determine a blood flow velocity according to the blood flow Doppler signal; and

a Doppler blood flow image generation module, configured to generate a Doppler blood flow image of the blood flow velocity measuring interest region according to the blood flow velocity, where the Doppler blood flow image is configured to display blood flow velocities at different spatial positions in the blood flow velocity measuring interest region.

In some embodiments, the inclination angle determination module may specifically include:

a first inclination angle determination unit, configured to determine, when the number of the transmitting sub-apertures is an even number, the inclination angle of the plane wave in each of the transmitting sub-apertures according to a formula

$${β}_{n} = \{ \begin{matrix}- arctan ( \frac{y}{\frac{1}{2} ( N - 2 n + 1 ) \times {L}_{s u b} \times {W}_{e l e m e n t}} ) & 1 \leq n \leq \frac{N}{2} \\ arctan ( \frac{y}{\frac{1}{2} ( 2 n - N - 1 ) \times {L}_{s u b} \times {W}_{e l e m e n t}} ) & \frac{N}{2} < n \leq N\end{matrix} ,$$

where, βₙ is the inclination angle of the plane wave in each of the transmitting sub-apertures; y is a vertical coordinate of the blood flow velocity measuring interest region; N is the number of the transmitting sub-apertures; n is a serial number of the transmitting sub-aperture, 1≤n≤N; L_{sub} is the number of array elements in each of the transmitting sub-apertures, and Wₑₗₑₘₑₙₜ is a width of an array element; and

a second inclination angle determination unit, configured to determine, when the number of the transmitting sub-apertures is an odd number, the inclination angle of the plane wave in each of the transmitting sub-apertures according to a formula

$${β}_{n} = \{ \begin{matrix}- arctan ( \frac{y}{\frac{1}{2} ( N - 2 n + 1 ) \times {L}_{s u b} \times {W}_{e l e m e n t}} ) & 1 \leq n < \frac{N + 1}{2} \\ 90 & n = \frac{N + 1}{2} \\ arctan ( \frac{y}{\frac{1}{2} ( 2 n - N - 1 ) \times {L}_{s u b} \times {W}_{e l e m e n t}} ) & \frac{N + 1}{2} < n \leq N\end{matrix} .$$

In some embodiments, the array element excitation delay time determination module may specifically include:

an array element excitation delay time determination unit, configured to determine the array element excitation delay time in each of the transmitting sub-apertures according to a formula

$$t ( {l}_{s u b} ) = \frac{( {l}_{s u b} - 1 ) \times {W}_{e lement} \times sin ( 90 ° - {β}_{n} )}{c} ,$$

where, t(l_{sub}) is the array element excitation delay time; c is a UTV in tissues; and l_{sub} is a serial number of an array element.

In some embodiments, the radio frequency signal sequence generation module may specifically include:

a compounded radio frequency signal generation unit, configured to perform beamforming on the echo signal with an ultrasound DAS method to generate a frame of compounded radio frequency signal for the blood flow velocity measuring interest region; and

a radio frequency signal sequence generation module, configured to generate the radio frequency signal sequence according to the compounded radio frequency signal.

In some embodiments, the blood flow velocity determination module may specifically include:

a blood flow velocity determination unit, configured to determine the blood flow velocity according to a formula

$$v = \frac{c {f}_{p}}{4 π {f}_{0}} \times φ ,$$

where, v is the blood flow velocity; φ is a phase shift of the blood flow Doppler signal; fₚ is a pulse repetition frequency, which equals to a frame rate of the compounded radio frequency signal; and f₀ is a central frequency of an ultrasound transducer.

According to specific embodiments of the present disclosure, the present disclosure discloses the following technical effects: The ultrasonic Doppler blood flow imaging method and system control, based on the inclination angle of the plane wave in each transmitting sub-aperture, all transmitting sub-apertures to simultaneously transmit the plane wave at multiple angles, thereby solving the problem of double loss of the pulse repetition frequency in CPWC imaging, and maximizing the pulse repetition frequency; and meanwhile, as multi-angle plane waves are simultaneously transmitted, the present disclosure avoids the motion artifacts in the imaging region of the CPWC, and suppresses the motion artifacts in the compounded radio frequency signal during ultrafast ultrasonic Doppler blood flow imaging.

### BRIEF DESCRIPTION OF THE DRAWINGS

The present disclosure will be further described below in combination with the accompanying drawings.

FIG. 1 is a flow chart of an ultrasonic Doppler blood flow imaging method according to the present disclosure.

FIG. 2 is a schematic view for calculating an inclination angle of a plane wave in a transmitting sub-aperture according to the present disclosure.

FIG. 3 is a schematic view of an imaging region of a CPWC according to the present disclosure.

FIG. 4 is a schematic view of a blood flow model according to the present disclosure.

FIG. 5 is a schematic view of parameter calculation according to the present disclosure.

FIG. 6 is a schematic view of a blood flow velocity imaging result according to the present disclosure.

FIG. 7 is a schematic structural view of an ultrasonic Doppler blood flow imaging system according to the present disclosure.

In the figures: 1. ultrasound transducer; 2. sub-aperture for transmitting a group of plane waves; 3. array element for transmitting a group of plane waves; 4. blood flow imaging interest region; 5. 0° plane wave; 6. plane wave having an inclination angle of β₁; 7. plane wave having an inclination angle of β₋₁; 8. blood flow velocity profile in specific embodiment; 9. central maximum velocity in blood flow velocity profile; and 10. inclination angle of blood vessel.

### DETAILED DESCRIPTION OF THE EMBODIMENTS

The technical solutions of the embodiments of the present disclosure are clearly and completely described below in combination with the accompanying drawings. Apparently, the described embodiments are merely a part rather than all of the embodiments of the present disclosure. All other examples obtained by a person of ordinary skill in the art based on the examples of the present disclosure without creative efforts shall fall within the protection scope of the present disclosure.

An objective of the present disclosure is to provide an ultrasonic Doppler blood flow imaging method and system, to maximize the pulse repetition frequency, and suppress the motion artifacts in the compounded radio frequency signal during ultrafast ultrasonic Doppler blood flow imaging.

To make the above-mentioned objectives, features, and advantages of the present disclosure clearer and more comprehensible, the present disclosure will be further described in detail below in combination with the accompanying drawings and the specific implementation.

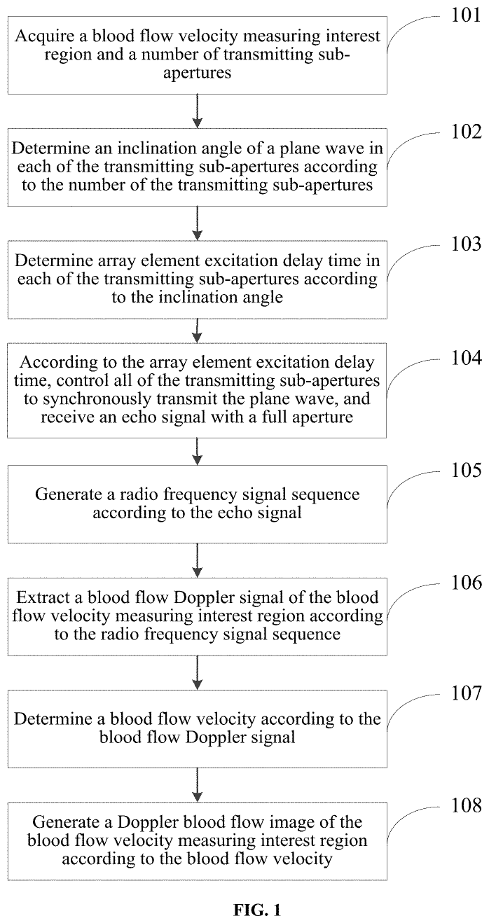

FIG. 1 is a flow chart of an ultrasonic Doppler blood flow imaging method according to the present disclosure. As shown in FIG. 1, the ultrasonic Doppler blood flow imaging method includes the following steps.

In Step 101, a blood flow velocity measuring interest region and the number of transmitting sub-apertures 2 are acquired.

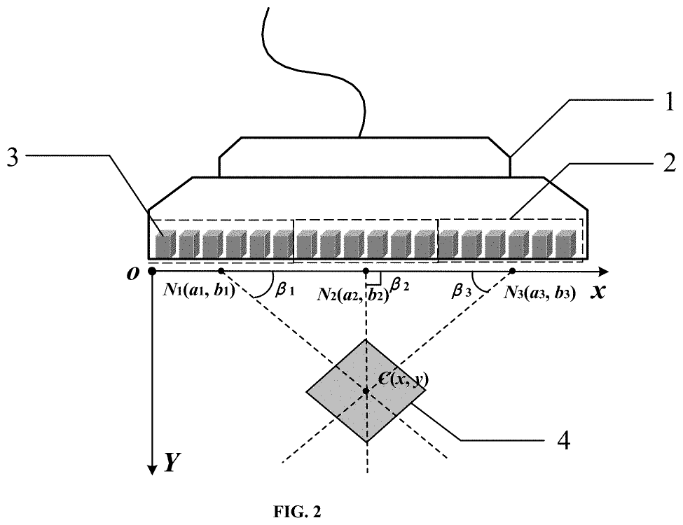

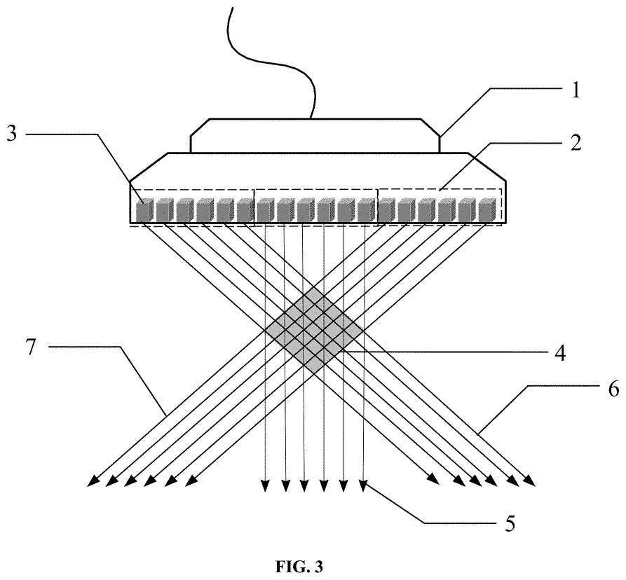

As shown in FIGS. 2-3, the blood flow imaging interest region 4 is selected.

In the region, the transverse range is xₐ˜x_{b}, with the transverse center being x=(x_{b}−xₐ)÷2; and the longitudinal range is yₐ˜y_{b}, with the longitudinal center being y=(y_{b}−yₐ)÷2.

the number N of transmitting sub-apertures 2 is set, and the number L_{sub} of array elements in each sub-aperture 2 is calculated:

$${L}_{s u b} = ⌊ \frac{{L}_{f u l l}}{N} ⌋$$

where, L_{full} is the total number of array elements of an ultrasound transducer 1, and [ . . . ] is rounded down.

In Step 102, an inclination angle of a plane wave in each transmitting sub-aperture 2 is determined according to the number of transmitting sub-apertures 2.

The inclination angle βₙ of the plane wave (specifically, the plane wave 5 having the inclination angle of 0°, the plane wave 6 having the inclination angle of β₁, and the plane wave 7 having the inclination angle of (β₋₁) in each transmitting sub-aperture 2, are calculated.

When the number N of transmitting sub-apertures 2 is an even number,

$${β}_{n} = \{ \begin{matrix}- arctan ( \frac{y}{\frac{1}{2} ( N - 2 n + 1 ) \times {L}_{s u b} \times {W}_{e l e m e n t}} ) & 1 \leq n \leq \frac{N}{2} \\ arctan ( \frac{y}{\frac{1}{2} ( 2 n - N - 1 ) \times {L}_{s u b} \times {W}_{e l e m e n t}} ) & \frac{N}{2} < n \leq N\end{matrix}$$

When the number N of transmitting sub-apertures 2 is an odd number,

$${β}_{n} = \{ \begin{matrix}- arctan ( \frac{y}{\frac{1}{2} ( N - 2 n + 1 ) \times {L}_{s u b} \times {W}_{e l e m e n t}} ) & 1 \leq n < \frac{N + 1}{2} \\ 90 & n = \frac{N + 1}{2} \\ arctan ( \frac{y}{\frac{1}{2} ( 2 n - N - 1 ) \times {L}_{s u b} \times {W}_{e l e m e n t}} ) & \frac{N + 1}{2} < n \leq N\end{matrix}$$

where, 1≤n≤N is a serial number of a sub-aperture 2, and Wₑₗₑₘₑₙₜ is a width of an array element.

In Step 103, array element excitation delay time in each transmitting sub-aperture 2 is determined according to the inclination angle.

The array element excitation delay time in each transmitting sub-aperture 2 is calculated.

The total number of array elements in each transmitting sub-aperture 2 is set as L_{sub}, and delay time for exciting an l_{sub}th array element 3 is calculated as:

$$t ( {l}_{s u b} ) = \frac{( {l}_{s u b} - 1 ) \times {W}_{e lement} \times sin ( 90 ° - {β}_{n} )}{c} \\ ( 1 \leq n \leq N , 1 \leq {l}_{s u b} \leq {L}_{s u b} )$$

In Step 104, according to the array element excitation delay time, all transmitting sub-apertures 2 are controlled to synchronously transmit the plane wave and to receive an echo signal with a full aperture.

Beamforming may be performed on the echo signal CD based on an ultrasound DAS method to generate a frame of compounded radio frequency signal RF(x,y) for the blood flow velocity measuring interest region.

RF(x,y)=∫ₓ₋ₐ^{x+a}CD(x′,t(x,y))dx′

where, 2a is the number of array elements used in the beamforming, xₐ≤x≤x_{b} and yₐ≤y≤y_{b}.

In Step 105, a radio frequency signal sequence is generated according to the echo signal.

Step 103-Step 104 are repeated for M times to obtain a time sequence RFₘ(l_{sub}, k) for M frames of compounded radio frequency signals, 1≤m≤M, 1≤l_{sub}≤L_{sub} and 1≤k≤K.

In the compounded radio frequency signal time sequence, there are M frames of compounded radio frequency signals. Each frame of compounded radio frequency signal includes L_{sub} lines of compounded radio frequency signals; and each line of compounded radio frequency signal includes K sampling points.

In Step 106, a blood flow Doppler signal of the blood flow velocity measuring interest region is extracted according to the radio frequency signal sequence.

blood flow Doppler signals B of all spatial positions may be extracted from the compounded radio frequency signal time sequence RFₘ(l_{sub}, k).

The blood flow Doppler signal at each spatial position includes M sampling points. The spatial position (l_{sub}, k) is used as an example to describe the extraction process of the blood flow Doppler signal B(l_{sub,k}):

B₍ₗ_{_{sub}}_{,k)}(m)=RFₘ(l_{sub},k)

where, 1≤m≤M.

In Step 107, a blood flow velocity may be determined according to the blood flow Doppler signal.

all blood flow Doppler signals are respectively demodulated:

B(m)=R(m)+j*l(m)

where, R(m) and l(m) are respectively an in-phase component and a quadrature component of the B(m) subjected to quadrature modulation, and j is an imaginary unit \sqrt{−1}.

A phase shift φ of the blood flow Doppler signal is calculated,

$$φ = \frac{{\sum }_{m = 2}^{M} I ( m ) R ( m - 1 ) - R ( m ) I ( m - 1 )}{{\sum }_{m = 2}^{M} R ( m ) R ( m - 1 ) + I ( m ) I ( m - 1 )}$$

Blood flow velocity information v at different radial positions is extracted according to a Doppler ultrasound formula,

$$v = \frac{c {f}_{p}}{4 π {f}_{0}} \times φ$$

where, c is a UTV in a human body tissue; fₚ is a pulse repetition frequency, which equals to a frame rate of the compounded radio frequency signal in ultrafast ultrasound; and f₀ is a central frequency of the ultrasound transducer 1.

In Step 108, a Doppler blood flow image of the blood flow velocity measuring interest region is generated according to the blood flow velocity, where the Doppler blood flow image is configured to display blood flow velocities at different spatial positions in the blood flow velocity measuring interest region.

A 2D blood flow velocity image in a field of view (FOV) is acquired according to blood flow velocity information v at all spatial positions in a color-coding interest region.

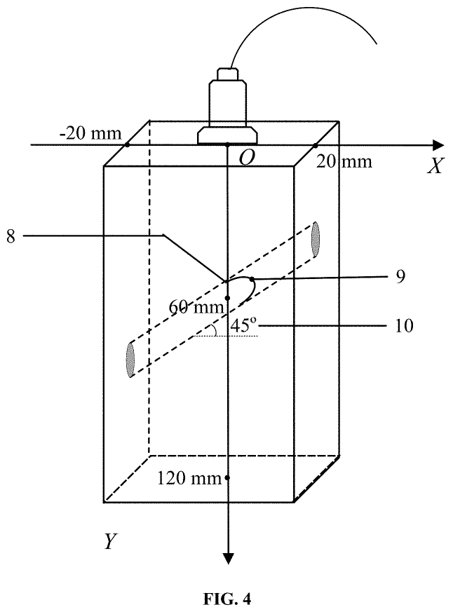

In actual applications, a blood flow model is established as shown in FIG. 4. In a case where the cylindrical blood vessel is located at 60 mm below the subcutaneous tissue, the lumen has a radius of R=5 mm, the blood vessel has an inclination angle 10 of 45° and a central maximum velocity 9 of vₘₐₓ=1.5 m/s, the blood flow velocity profile 8 from the upper vessel wall to the lower vessel wall is

$$v ( r ) = {v}_{max} ( 1 - \frac{{r}^{2}}{{R}^{2}} ) , where , 1 \leq r \leq R .$$

The blood flow imaging interest region 4 is selected. The transverse range is xₐ=−6 mm to x_{b}=6 mm, with the transverse center being x=(x_{b}−xₐ)÷2=0 mm; and the longitudinal range is yₐ=50 mm to y_{b}=70 mm, with the longitudinal center being y=(y_{b}−yₐ)÷2=60 mm.

The number N=3 of transmitting sub-apertures 2 is set, and the number L_{sub}=42 of array elements in each sub-aperture 2 is calculated as:

$${L}_{s u b} = ⌊ \frac{{L}_{f u l l}}{N} ⌋ = 42$$

where, L_{full}=128, indicating the total number of array elements of an ultrasound transducer 1, and [ . . . ] means rounding down.

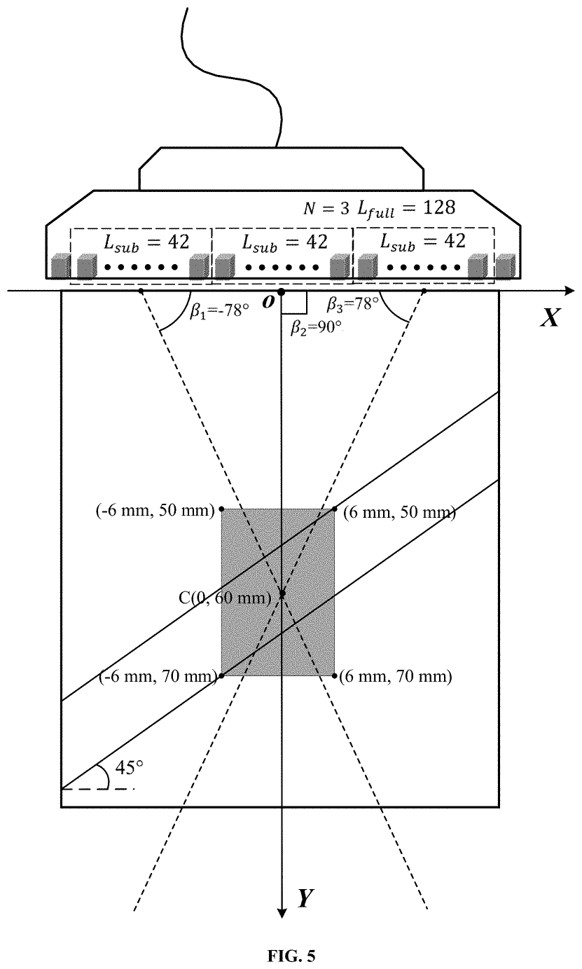

As shown in FIG. 5, an inclination angle βₙ of a plane wave in each transmitting sub-aperture 2 is calculated. When the number N=3 of transmitting sub-apertures 2 is an odd number, the number is substituted into the following formula to calculate the inclination angle βₙ.

$${β}_{n} = \{ \begin{matrix}- arctan ( \frac{y}{\frac{1}{2} ( N - 2 n + 1 ) \times {L}_{s u b} \times {W}_{e l e m e n t}} ) & 1 \leq n < \frac{N + 1}{2} \\ 90 & n = \frac{N + 1}{2} \\ arctan ( \frac{y}{\frac{1}{2} ( 2 n - N - 1 ) \times {L}_{s u b} \times {W}_{e l e m e n t}} ) & \frac{N + 1}{2} < n \leq N\end{matrix}$$

where, 1≤n≤N is a serial number of a sub-aperture 2, and Wₑₗₑₘₑₙₜ=0.3 mm is a width of an array element. β₁=−78°, β₂=90° and β₃=78°.

Array element excitation delay time in each sub-aperture 2 is calculated according to the inclination angle β₁=−78°, β₂=90° and β₃=78°.

The total number of array elements in each transmitting sub-aperture 2 set as

L_{sub}=42, and delay time for exciting an l_{sub}th array element 3 is calculated as:

$$t ( {l}_{s u b} ) = \frac{( {l}_{s u b} - 1 ) \times {W}_{e l e m e n t} \times sin ( 90 ° - {β}_{n} )}{c} \\ ( 1 \leq n \leq 3 , 1 \leq {l}_{s u b} \leq 42 )$$

According to the array element excitation delay time, all sub-apertures 2 are controlled to synchronously transmit the plane wave.

An echo signal CD is received with a full aperture.

Beamforming may be performed on the echo signal CD based on an ultrasound DAS method to generate a frame of compounded radio frequency signal RF(x,y) for the blood flow velocity measuring interest region.

RF(x,y)=∫ₓ₋ₐ^{x+a}CD(x′,t(x,y))dx′

where, 2a is the number of array elements used in the beamforming, −6mm≤x≤6mm and 50 mm≤y≤70 mm.

Step of “calculating an inclination angle βₙ of a plane wave in each transmitting sub-aperture 2” to Step of “Receiving an echo signal CD with a full aperture” are repeated, the number M of repetitions is equal to 10, to obtain a time sequence RFₘ(l_{sub}, k) for 10 frames of compounded radio frequency signals, 1≤m≤10, 1≤l_{sub}≤42 and 1≤k≤650.

In the compounded radio frequency signal time sequence, there are M=10 frames of compounded radio frequency signals. Each frame of compounded radio frequency signal includes L_{sub}=42 lines of compounded radio frequency signals; and each line of compounded radio frequency signal includes K=650 sampling points.

blood flow Doppler signals B of all spatial positions are extracted from the compounded radio frequency signal time sequence RFₘ(l_{sub}, k).

The blood flow Doppler signal at each spatial position includes M=10 sampling points.

The spatial position (l_{sub}, k) is taken as an example to describe the extraction process of the blood flow Doppler signal B(l_{sub},k):

B₍ₗ_{_{sub}}_{,k)}(m)=RFₘ(l_{sub},k)

where, 1≤m≤10.

All blood flow Doppler signals are respectively demodulated,

B(m)=R(m)+j*l(m)

where, R(k) and B(k) are respectively an in-phase component and a quadrature component of the R(k) subjected to quadrature modulation, and j is an imaginary unit \sqrt{−1}.

A phase shift φ of the blood flow Doppler signal is calculated,

$$φ = arctan \frac{{\sum }_{m = 2}^{M} I ( m ) R ( m - 1 ) - R ( m ) I ( m - 1 )}{{\sum }_{m = 2}^{M} R ( m ) R ( m - 1 ) + I ( m ) I ( m - 1 )}$$

Blood flow velocity information v at different spatial positions in the blood flow imaging interest region 4 is extracted according to a Doppler ultrasound formula:

$$v = \frac{c {f}_{p}}{4 π {f}_{0}} \times φ$$

where, c is a UTV in a human body tissue and is typically 1,540 m/s in the human body tissue, fₚ=20,000 Hz is a pulse repetition frequency, and f₀=10 MHz is a central frequency of the ultrasound transducer 1.

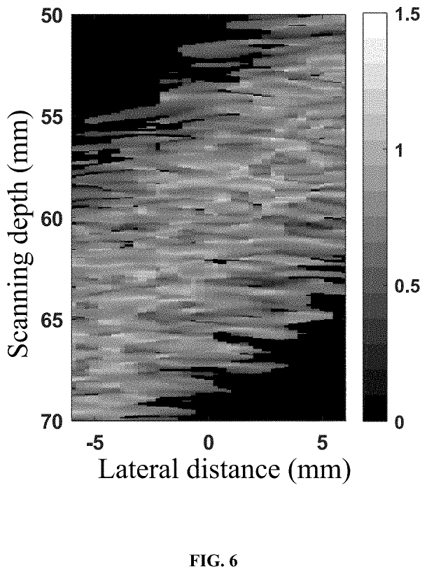

A 2D blood flow velocity image in an FOV is acquired according to blood flow velocity information v at all spatial positions in the color-coding interest region, with a result as shown in FIG. 6.

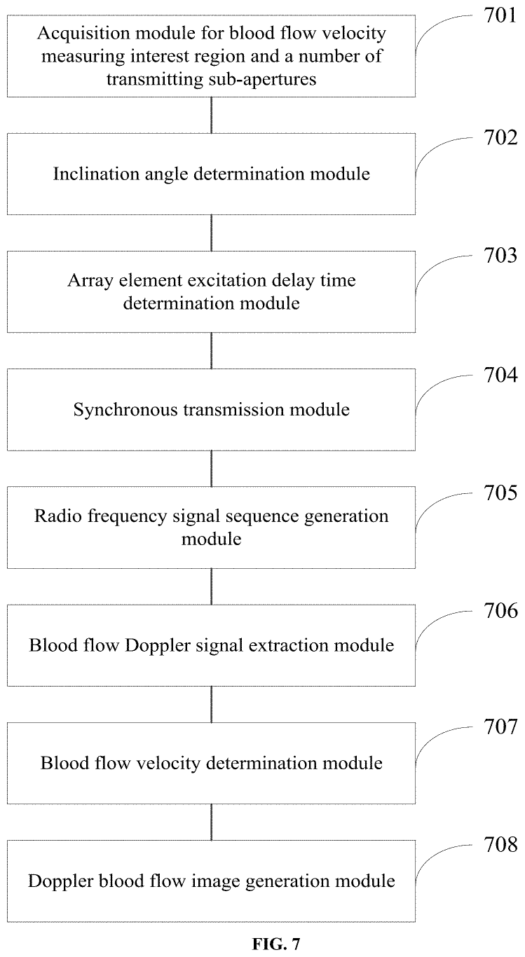

FIG. 7 is a schematic structural view of an ultrasonic Doppler blood flow imaging system according to the present disclosure. As shown in FIG. 7, the ultrasonic Doppler blood flow imaging system includes: an acquisition module 701 for a blood flow velocity measuring interest region and the number of transmitting sub-apertures 2, an inclination angle determination module 702, an array element excitation delay time determination module 703, a synchronous transmission module 704, a radio frequency signal sequence generation module 705, a blood flow Doppler signal extraction module 706, a blood flow velocity determination module 707, and a Doppler blood flow image generation module 708.

The acquisition module 701 for a blood flow velocity measuring interest region and the number of transmitting sub-apertures 2 is configured to acquire the blood flow velocity measuring interest region and the number of transmitting sub-apertures 2.

The inclination angle determination module 702 is configured to determine an inclination angle of a plane wave in each transmitting sub-aperture 2 according to the number of transmitting sub-apertures 2.

The inclination angle determination module 702 specifically includes:

a first inclination angle determination unit, configured to determine, when the number of transmitting sub-apertures 2 is an even number, the inclination angle of the plane wave in each transmitting sub-aperture 2 according to a formula

$${β}_{n} = \{ \begin{matrix}- arctan ( \frac{y}{\frac{1}{2} ( N - 2 n + 1 ) \times {L}_{s u b} \times {W}_{e l e m e n t}} ) & 1 \leq n \leq \frac{N}{2} \\ arctan ( \frac{y}{\frac{1}{2} ( 2 n - N - 1 ) \times {L}_{s u b} \times {W}_{e l e m e n t}} ) & \frac{N}{2} < n \leq N\end{matrix} ,$$

where, βₙ is the inclination angle of the plane wave in each transmitting sub-aperture 2; y is a vertical coordinate of the blood flow velocity measuring interest region; N is the number of transmitting sub-apertures 2; n is a serial number of a transmitting sub-aperture 2, 1≤n≤N; L_{sub} is the number of array elements in each transmitting sub-aperture 2; and Wₑₗₑₘₑₙₜ is a width of an array element; and

a second inclination angle determination unit, configured to determine, when the number of transmitting sub-apertures 2 is an odd number, the inclination angle of the plane wave in each transmitting sub-aperture 2 according to a formula

$${β}_{n} = \{ \begin{matrix}- arctan ( \frac{y}{\frac{1}{2} ( N - 2 n + 1 ) \times {L}_{s u b} \times {W}_{e l e m e n t}} ) & 1 \leq n < \frac{N + 1}{2} \\ 90 & n = \frac{N + 1}{2} \\ arctan ( \frac{y}{\frac{1}{2} ( 2 n - N - 1 ) \times {L}_{s u b} \times {W}_{e l e m e n t}} ) & \frac{N + 1}{2} < n \leq N\end{matrix} .$$

The array element excitation delay time determination module 703 is configured to determine array element excitation delay time in each transmitting sub-aperture 2 according to the inclination angle.

The array element excitation delay time determination module 703 specifically includes:

an array element excitation delay time determination unit, configured to determine the array element excitation delay time in each transmitting sub-aperture 2 according to a formula

$$t ( {l}_{s u b} ) = \frac{( {l}_{s u b} - 1 ) \times {W}_{e l e m e n t} \times sin ( 90 ° - {β}_{n} )}{c} ,$$

where, t(l_{sub}) is the array element excitation delay time; c is a UTV in a human body tissue; and l_{sub} is a serial number of an array element.

The synchronous transmission module 704 is configured to control, according to the array element excitation delay time, all of the transmitting sub-apertures 2 to synchronously transmit the plane wave, and receive an echo signal with a full aperture.

The radio frequency signal sequence generation module 705 is configured to generate a radio frequency signal sequence according to the echo signal.

The radio frequency signal sequence generation module 705 specifically includes:

a compounded radio frequency signal generation unit, configured to perform beamforming on the echo signal with an ultrasound DAS method to generate a frame of compounded radio frequency signal for the blood flow velocity measuring interest region; and

a radio frequency signal sequence generation module, configured to generate the radio frequency signal sequence according to the compounded radio frequency signal.

The blood flow Doppler signal extraction module 706 is configured to extract a blood flow Doppler signal of the blood flow velocity measuring interest region according to the radio frequency signal sequence.

The blood flow velocity determination module 707 is configured to determine a blood flow velocity according to the blood flow Doppler signal.

The blood flow velocity determination module 707 specifically includes:

a blood flow velocity determination unit, configured to determine the blood flow velocity according to a formula

$$v = \frac{c {f}_{p}}{4 π {f}_{0}} \times φ ,$$

where, v is the blood flow velocity; φ is a phase shift of the blood flow Doppler signal; fₚ is a pulse repetition frequency, which equals to a frame rate of the compounded radio frequency signal; and f₀ is a central frequency of an ultrasound transducer 1.

The Doppler blood flow image generation module 708 is configured to generate a Doppler blood flow image of the blood flow velocity measuring interest region according to the blood flow velocity, where the Doppler blood flow image is configured to display blood flow velocities at different spatial positions in the blood flow velocity measuring interest region.

In accordance with the present disclosure, the number of array elements in each sub-aperture 2 and the inclination angle of the plane wave are calculated according to the blood flow velocity measuring interest region and the preset number of sub-apertures 2, and the plane waves at multiple angles are synchronously transmitted according to the inclination angle of each plane wave, thereby maximizing the pulse repetition frequency, and suppressing the motion artifacts in the compounded radio frequency signal during ultrafast ultrasonic Doppler blood flow imaging.

The above embodiments are provided merely for an objective of describing the present disclosure and are not intended to limit the scope of the present disclosure. The scope of the present disclosure is defined by the appended claims. Various equivalent replacements and modifications made without departing from the spirit and scope of the present disclosure should all fall within the scope of the present disclosure.

What is claimed is:

1. An ultrasonic Doppler blood flow imaging method, comprising:
acquiring a blood flow velocity measuring interest region and a number of transmitting sub-apertures;
determining an inclination angle of a plane wave in each of the transmitting sub-apertures according to the number of the transmitting sub-apertures;
determining array element excitation delay time in each of the transmitting sub-apertures according to the inclination angle;
controlling, according to the array element excitation delay time, all of the transmitting sub-apertures to synchronously transmit the plane wave, and receiving an echo signal with a full aperture;
generating a radio frequency signal sequence according to the echo signal;
extracting a blood flow Doppler signal of the blood flow velocity measuring interest region according to the radio frequency signal sequence;
determining a blood flow velocity according to the blood flow Doppler signal; and
generating a Doppler blood flow image of the blood flow velocity measuring interest region according to the blood flow velocity, wherein the Doppler blood flow image is configured to display blood flow velocities at different spatial positions in the blood flow velocity measuring interest region.

2. The ultrasonic Doppler blood flow imaging method according to claim 1, wherein the determining an inclination angle of a plane wave in each of the transmitting sub-apertures according to the number of the transmitting sub-apertures specifically comprises:
determining, when the number of the transmitting sub-apertures is an even number, the inclination angle of the plane wave in each of the transmitting sub-apertures according to a formula
$${β}_{n} = \{ \begin{matrix}- arctan ( \frac{y}{\frac{1}{2} ( N - 2 n + 1 ) \times {L}_{s u b} \times {W}_{e l e m e n t}} ) & 1 \leq n \leq \frac{N}{2} \\ arctan ( \frac{y}{\frac{1}{2} ( 2 n - N - 1 ) \times {L}_{s u b} \times {W}_{e l e m e n t}} ) & \frac{N}{2} < n \leq N\end{matrix} ,$$
wherein, βₙ is the inclination angle of the plane wave in each of the transmitting sub-apertures; y is a vertical coordinate of the blood flow velocity measuring interest region; N is the number of the transmitting sub-apertures; n is a serial number of the transmitting sub-aperture, 1≤n≤N; L_{sub} is the number of array elements in each of the transmitting sub-apertures; and Wₑₗₑₘₑₙₜ is a width of an array element; and
determining, when the number of the transmitting sub-apertures is an odd number, the inclination angle of the plane wave in each of the transmitting sub-apertures according to a formula
$${β}_{n} = \{ \begin{matrix}- arctan ( \frac{y}{\frac{1}{2} ( N - 2 n + 1 ) \times {L}_{s u b} \times {W}_{e l e m e n t}} ) & 1 \leq n < \frac{N + 1}{2} \\ 90 & n = \frac{N + 1}{2} \\ arctan ( \frac{y}{\frac{1}{2} ( 2 n - N - 1 ) \times {L}_{s u b} \times {W}_{e l e m e n t}} ) & \frac{N + 1}{2} < n \leq N\end{matrix} .$$

3. The ultrasonic Doppler blood flow imaging method according to claim 2, wherein the determining array element excitation delay time in each of the transmitting sub-apertures according to the inclination angle specifically comprises:
determining the array element excitation delay time in each of the transmitting sub-apertures according to a formula
$$t ( {l}_{s u b} ) = \frac{( {l}_{s u b} - 1 ) \times {W}_{e l e m e n t} \times sin ( 90 ° - {β}_{n} )}{c} ,$$
wherein, t(l_{sub}) is the array element excitation delay time; c is an ultrasound transmission velocity (UTV) in a human body tissue; and l_{sub} is a serial number of an array element.

4. The ultrasonic Doppler blood flow imaging method according to claim 3, wherein the generating a radio frequency signal sequence according to the echo signal specifically comprises:
performing beamforming on the echo signal with an ultrasound delay-and-sum (DAS) method to generate a frame of compounded radio frequency signal for the blood flow velocity measuring interest region; and
generating the radio frequency signal sequence according to the compounded radio frequency signal.

5. The ultrasonic Doppler blood flow imaging method according to claim 4, wherein the determining a blood flow velocity according to the blood flow Doppler signal specifically comprises:
determining the blood flow velocity according to a formula
$$v = \frac{c {f}_{p}}{4 π {f}_{0}} \times φ ,$$
wherein, v is the blood flow velocity; φ is a phase shift of the blood flow Doppler signal; fₚ is a pulse repetition frequency, which equals to a frame rate of the compounded radio frequency signal; and f₀ is a central frequency of an ultrasound transducer.

6. An ultrasonic Doppler blood flow imaging system, comprising:
an acquisition module for a blood flow velocity measuring interest region and a number of transmitting sub-apertures, configured to acquire the blood flow velocity measuring interest region and a number of transmitting sub-apertures;
an inclination angle determination module, configured to determine an inclination angle of a plane wave in each of the transmitting sub-apertures according to the number of the transmitting sub-apertures;
an array element excitation delay time determination module, configured to determine array element excitation delay time in each of the transmitting sub-apertures according to the inclination angle;
a synchronous transmission module, configured to control, according to the array element excitation delay time, all of the transmitting sub-apertures to synchronously transmit the plane wave, and receive an echo signal with a full aperture;
a radio frequency signal sequence generation module, configured to generate a radio frequency signal sequence according to the echo signal;
a blood flow Doppler signal extraction module, configured to extract a blood flow Doppler signal of the blood flow velocity measuring interest region according to the radio frequency signal sequence;
a blood flow velocity determination module, configured to determine a blood flow velocity according to the blood flow Doppler signal; and
a Doppler blood flow image generation module, configured to generate a Doppler blood flow image of the blood flow velocity measuring interest region according to the blood flow velocity, wherein the Doppler blood flow image is configured to display blood flow velocities at different spatial positions in the blood flow velocity measuring interest region.

7. The ultrasonic Doppler blood flow imaging system according to claim 6, wherein the inclination angle determination module specifically comprises:
a first inclination angle determination unit, configured to determine, when the number of the transmitting sub-apertures is an even number, the inclination angle of the plane wave in each of the transmitting sub-apertures according to a formula
$${β}_{n} = \{ \begin{matrix}- arctan ( \frac{y}{\frac{1}{2} ( N - 2 n + 1 ) \times {L}_{s u b} \times {W}_{e l e m e n t}} ) & 1 \leq n \leq \frac{N}{2} \\ arctan ( \frac{y}{\frac{1}{2} ( 2 n - N - 1 ) \times {L}_{s u b} \times {W}_{e l e m e n t}} ) & \frac{N}{2} < n \leq N\end{matrix} ,$$
wherein, βₙ is the inclination angle of the plane wave in each of the transmitting sub-apertures; y is a vertical coordinate of the blood flow velocity measuring interest region; N is the number of the transmitting sub-apertures; n is a serial number of the transmitting sub-aperture, 1≤n≤N; L_{sub} is the number of array elements in each of the transmitting sub-apertures; and W element is a width of an array element; and
a second inclination angle determination unit, configured to determine, when the number of the transmitting sub-apertures is an odd number, the inclination angle of the plane wave in each of the transmitting sub-apertures according to a formula
$${β}_{n} = \{ \begin{matrix}- arctan ( \frac{y}{\frac{1}{2} ( N - 2 n + 1 ) \times {L}_{s u b} \times {W}_{e l e m e n t}} ) & 1 \leq n < \frac{N + 1}{2} \\ 90 & n = \frac{N + 1}{2} \\ arctan ( \frac{y}{\frac{1}{2} ( 2 n - N - 1 ) \times {L}_{s u b} \times {W}_{e l e m e n t}} ) & \frac{N + 1}{2} < n \leq N\end{matrix} .$$

8. The ultrasonic Doppler blood flow imaging system according to claim 7, wherein the array element excitation delay time determination module specifically comprises:
an array element excitation delay time determination unit, configured to determine the array element excitation delay time in each of the transmitting sub-apertures according to a formula
$$t ( {l}_{s u b} ) = \frac{( {l}_{s u b} - 1 ) \times {W}_{e l e m e n t} \times sin ( 90 ° - {β}_{n} )}{c} ,$$
wherein, t(l_{sub}) is the array element excitation delay time; c is an ultrasound transmission velocity (UTV) in a human body tissue; and l_{sub} is a serial number of an array element.

9. The ultrasonic Doppler blood flow imaging system according to claim 8, wherein the radio frequency signal sequence generation module specifically comprises:
a compounded radio frequency signal generation unit, configured to perform beamforming on the echo signal with an ultrasound delay-and-sum (DAS) method to generate a frame of compounded radio frequency signal for the blood flow velocity measuring interest region; and
a radio frequency signal sequence generation module, configured to generate the radio frequency signal sequence according to the compounded radio frequency signal.

10. The ultrasonic Doppler blood flow imaging system according to claim 9, wherein the blood flow velocity determination module specifically comprises:
a blood flow velocity determination unit, configured to determine the blood flow velocity according to a formula
$$v = \frac{c {f}_{p}}{4 π {f}_{0}} \times φ ,$$
wherein, v is the blood flow velocity; φ is a phase shift of the blood flow Doppler signal; fₚ is a pulse repetition frequency, which equals to a frame rate of the compounded radio frequency signal; and f₀ is a central frequency of an ultrasound transducer.
